(19) United States
(12) Patent Application Publication
(10) Pub. No.: US 2008/0009897 A1
(43) Pub. Date: Jan. 10, 2008

(54) Nasal Stimulator

(75) Inventor: Josep Duran Von Arx, Barcelona (ES)

Correspondence Address: DEFILLO & ASSOCIATES, INC., P.O. Box 14104, Clearwater, FL, 33766, US

(73) Assignee: Institute Orthodontic World J. Duran Von Arx, S.L., Barcelona (ES)

(21) Appl. No.: 10/573,183

(22) PCT Filed: Sep. 22, 2004
(86) PCT No.: PCT/ES04/00416

§ 371 Date: Mar. 23, 2006

(30) Foreign Application Priority Data
Sep. 22, 2003 (ES) P200302195

(51) Int. Cl. A61M 29/00; A61F 5/08

(52) U.S. Cl. 606/199; 128/200.24

(57) ABSTRACT

The invention relates to a nasal stimulator which is used to dilate and stimulate the elevator muscle of the nose wing. The invention is designed to improve nasal breathing in persons suffering from nasal collapse, who, consequently, breathe mainly through the mouth.

### OBJECTIVE OF THE INVENTION

Nasal stimulator whose function is to dilate and stimulate the levator muscle of the alar sidewall of the nose to improve breathing for persons who suffer nasal blockage hence their breathing is done mainly through the mouth. This invention alleviates this problem by inserting a cylinder with interior perforations made of silicon, into one or each nostril, and when a dilation is produced in the nostril, it eases breathing.

### BACKGROUND OF THE INVENTION

Discomfort or slight pain occurs because of incorrect breathing through the nose. This pain and/or discomfort is the result of different motives, such as having a deviated septum, lack of stimulation of the nasal muscle, having defective nasal cartilage, etc.

There are many different products in the market which solve breathing problems that people with a defective nasal structure have. These are applied by inserting them in the nostrils either in liquid form or in spray, with the objective of causing the nasal muscle to dilate therefore, allowing normal breathing for a certain period of time.

The actual invention avoids the use of any type of liquid or spray in the nostrils, since these products are less effective in the long run and can cause a kind of addiction to them. On the contrary, the actual invention of the nasal stimulator acts by inserting in one or each nostril, depending on each individuals needs, an internally perforated silicon cylinder with a featured widened periphery except in the area which faces the nasal septum or wall which dilates the opening space or dome, stimulates the levator muscle of the nasal alar and helps the nasal wall or septum center remodel the nasal cartilage; the said cylinder also contains a protruding lip on its lower rim which has contact with the external part of the alar sidewall of the nose and stimulates the levator muscle of the noses' alar sidewall through the exterior. The summary of the anterior explanation is that the said stimulator facilitates the users breathing through the nose.

This present invention of the nasal stimulator is designed for use by all the persons with nasal breathing problems, which could include athletes and individuals with general orthodontic problems.

### GENERAL DESCRIPTION OF THE INVENTION

The invented nasal stimulator is composed of one or two silicon cylinders of approximately 1 cm in height which are perforated in their interior and which widen in the central part of the external surface. This widening is generally peripheral except in the area of the cylinder which faces the septum wall. The grazing or touch that occurs between the said widening and the internal part of the nose stimulates the levator muscle of the nasal alar and the widening also causes a slight enlargement of the nasal orifice.

The lower part of each silicon cylinder ends in a lip or rim to impede the cylinders from going in the nostrils further than advisable when they are inserted in the nose to dilate the noses' opening space or domes. A type of support that jutts out is integrated onto this rim which makes contact with the external part of the alar of the nose and stimulates the levator muscle of the nasal alar through the exterior side of the same, it adds pressure on the exterior of the users nose and allows the joining and fastening of the nasal stimulator.

The joint effect of the internal grazing of the widened part to the internal side of the nostril, and the external grazing or touch of the protruding support with the external part of the alar of the nose, and consequently the dilation of the nostrils facilitates therefore, the comfortable breathing of the patient.

### BRIEF DESCRIPTION OF THE DRAWINGS

For a brief understanding of the objective of the said invention it is described as a practical realization of the invention around the basis of the attached figures where:

FIG. 1 shows a perspective view of the one cylinder invention of the nasal stimulator

FIG. 2 shows a view from below of the one cylinder invention of the nasal stimulator.

FIG. 3 shows a view from above of the one cylinder invention of the nasal stimulator.

FIG. 4 shows a section as in pattern E-E of FIG. 3.

FIG. 5 shows a perspective view of a double cylinder invention of the nasal stimulator

FIG. 6 shows a view from below of the nasal stimulator of FIG. 5.

FIG. 7 shows a view from above of the nasal stimulator of FIG. 5.

FIG. 8 shows a side view of the nasal stimulator of FIG. 5.

### PRINCIPAL EMBODIMENTS OF THE INVENTION

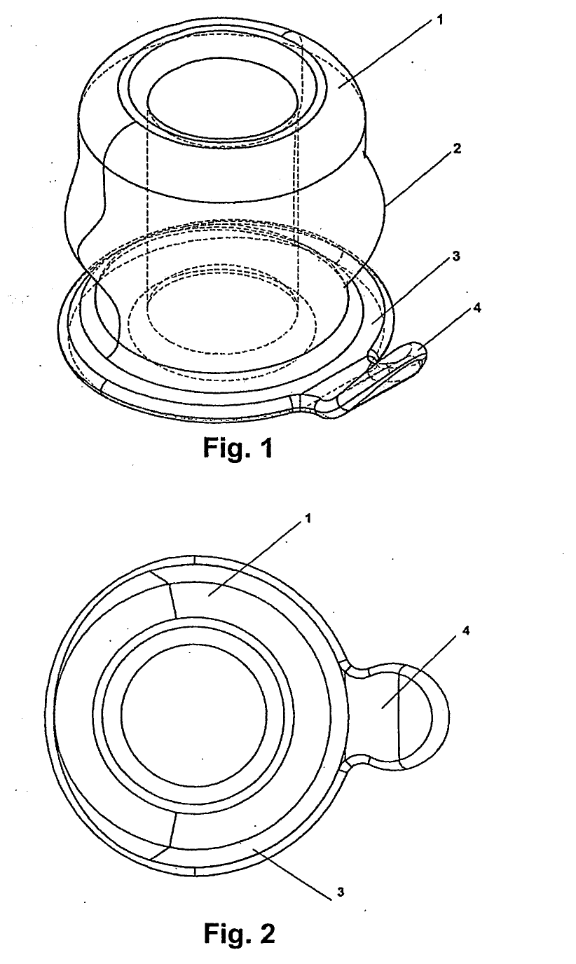

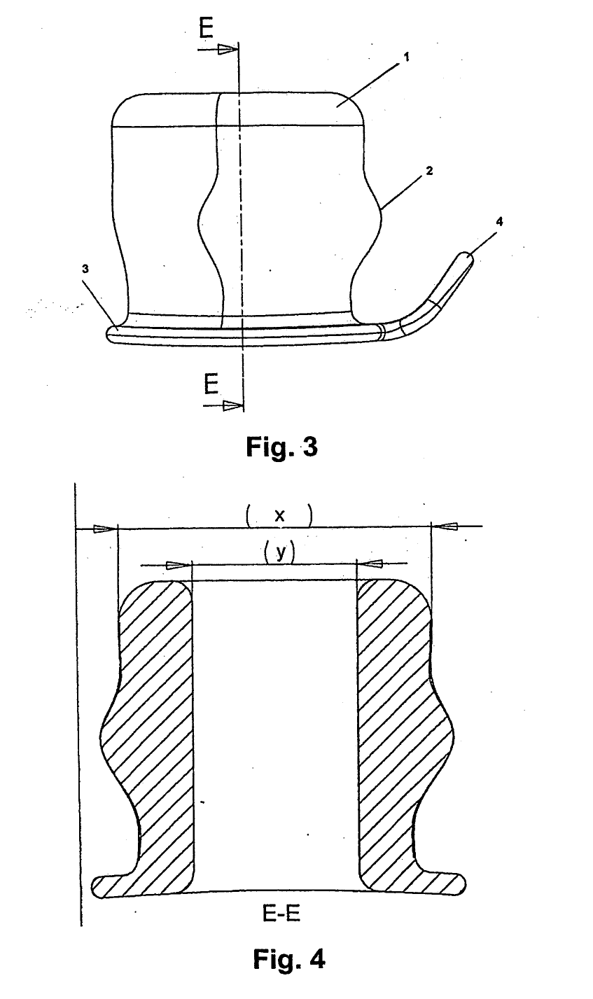

In an initial embodiment, the nasal stimulator invented is composed of, according to what is seen in FIGS. 1-4, a cylinder (1), perforated inside and made of silicon. Approximately halfway its exterior perimeter height, the cylinder widens in shape (2) throughout its whole periphery with the exception of the same area which faces the septum wall, whose function is to stimulate the levator muscle of the alar of the nose through the internal side of the nostril. This stimulation is caused by the touch or “grazing” that is produced between the widened part (2) and the interior of the nose which results in easing the respiration or breathing. In the lower part of the cylinder or the part that is left out when the user inserts the stimulator in the nose, the cylinder (1) has a peripheral rim (3) which serves as a limit where the insertion of the stimulator into the nose should not pass. Integrated onto this rim (3) is a protruding support or lip (4) which adds pressure on the exterior of the users nose and allows for the coupling or joining and fastening of the nasal stimulator. Moreover, and of utmost importance, the contact and touch with the external alar of the nose stimulates the levator muscle of the alar through the exterior of the same.

For a second embodiment, the nasal stimulator is composed of 2 identical and independent cylinders and one is inserted in each nostril. Although the dimensions are not limitative on the nature of the invention and should be in accordance with the users nose dimensions, some orientative dimensions are: it is of 1 cm in height, 8 mm in interior diameter ((Y) see FIG. 4) and between 12-16 mm in exterior diameter ((X) see FIG. 4)

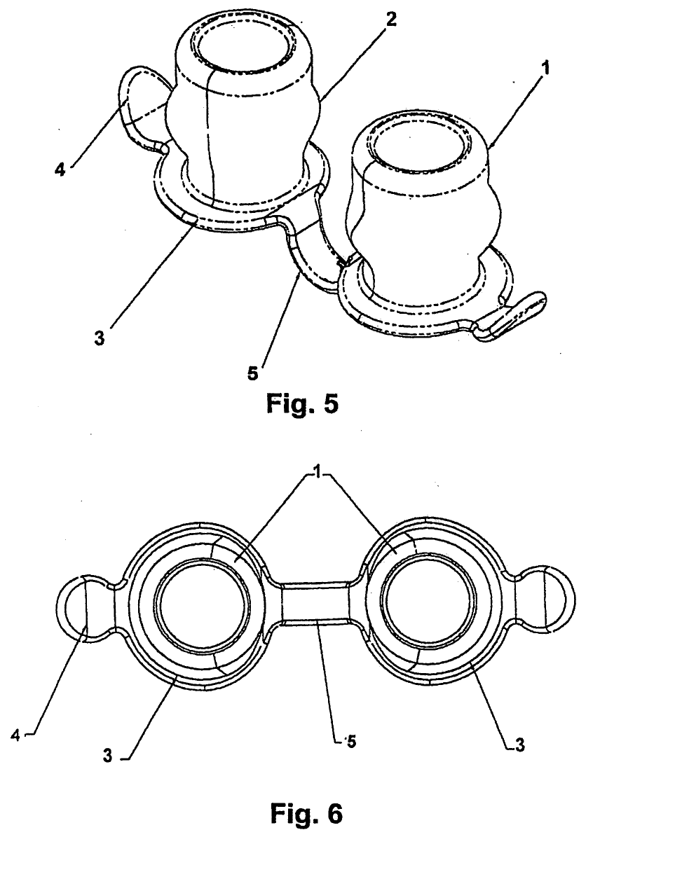

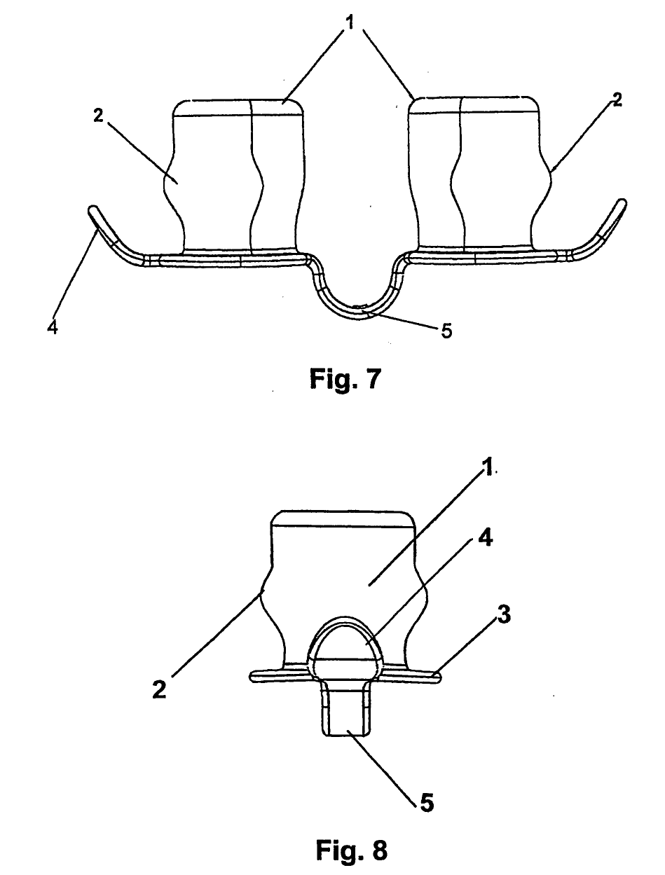

For a third embodiment, see FIG. 5-8, we can see the invented nasal stimulator formed by 2 cylinders (1),with their widenings (2) in the periphery of the cylinders except in the part of each which faces the nasal septum that is joined in the center by a tongue (5) that jutts out from the rims (3) in the part that is diametrically opposite the protruding supports (4) of each cylinder. The length of this tongue is enough so that one can insert both cylinders into the nostrils with the said tongue (5) slightly arched to facilitate its easy positioning.

Once the actual inventions nature is sufficiently described just as some main embodiments, it should be added that on its whole and on the parts that compose it, it is possible to introduce changes in shape, material and disposition, as long as the said alterations do not substantially vary the characteristics of the invention which are claimed heretofore.

1. Nasal stimulator made of silicon material which consists of one cylinder (1) internally perforated, or of two cylinders (1) joined by an curved tongue (5) displaying said cylinder/s (1) a rim on its lower part (3) that keeps the cylinder from being inserted into the nostril deeper than it should, with the said rim (3) having a protruding support or tab (4), characterized in that
it presents of a widening (2) in the central part of its external surface, with said widening covering its' whole periphery except in the small part that faces or comes into contact with the nasal septum during its use, which produces a touch or grazing on the internal wall of the nose that stimulates the levator muscle of the alar of the nose, and on where the mentioned support or protruding tab (4) causes a touch or grazing on the external wall of the nose that stimulates the levator muscle of the alar of the nose.
Therefore both morphological characteristics of the nasal stimulator, produce a grazing on the nose alar and as a consequence it stimulates the levator muscle of the nose.
